(19) United States
(12) Patent Application Publication
(10) Pub. No.: US 2022/0187307 A1
(43) Pub. Date: Jun. 16, 2022

(54) Biomarkers for improving nutrion for infants at risk

(71) Applicant: N.V. Nutricia, Zoetermeer (NL)

(72) Inventors: Hugo Philemon van Bever, Singapore (SG); Elena Sandalova, Singapore (SG); Alma Jildou Nauta, Utrecht (NL)

(73) Assignee: N.V. Nutricia, Zoetermeer (NZ)

(21) Appl. No.: 17/056,460

(22) PCT Filed: May 21, 2019
(86) PCT No.: PCT/EP2019/063103

§ 371 Date: Nov. 18, 2020

(30) Foreign Application Priority Data
May 22, 2018 (EP) 18173688.5

(51) Int. Cl. G01N 33/68

(52) U.S. Cl. CPC G01N 33/6854; G01N 2800/50; G01N 2800/38; G01N 2800/24

(57) ABSTRACT

The invention relates to biomarkers in the umbilical cord epithelium relating to skin proteins that are better predictive for the development of atopic dermatitis late in life. These biomarkers enable an early nutritional intervention in a more precisely determined population of at risk infants.

### FIELD OF THE INVENTION

The current invention is in the field of infant nutrition, in particular infant nutrition for infants at risk of developing atopic dermatitis.

### BACKGROUND OF THE INVENTION

Atopic dermatitis (AD) is a chronic inflammatory skin disease posing a significant burden on health-care resources and patients' quality of life. It is a complex disease with a wide spectrum of clinical presentations and combinations of symptoms. AD affects up to 20% of children and up to 3% of adults; recent data show that its prevalence is still increasing, especially in low-income countries. First manifestations of AD usually appear early in life and often precede other allergic diseases such as food allergy, asthma or allergic rhinitis. Fifty percent of all those with AD develop other allergic symptoms within their first year of life and probably as many as 85% of the patients experience an onset below 5 years of age. It is advantageous that prevention of AD can start as soon as possible after birth.

For infants suffering from allergy or atopic dermatitis several formulae are on the market comprising ingredients adapted treat the atopic diseases, such as allergy, in particular hydrolysed proteins which have a reduced allergenicity or formula with free amino acids, thereby treating the allergy be avoiding exposure to allergens. Infants born from parents of whom one or both suffers from an atopic disease, are considered to have a higher risk of developing an atopic disease. For this group, besides the preferred breast feeding, several infant formulae have been developed. For example, hypoallergenic formulae are available on the market, comprising a partial protein hydrolysate (partially hydrolysed proteins), which were shown to reduce the incidence of AD (Alexander and Cabana, 2010, JPGN; 50: 422-430). Also other ingredients have been demonstrated to have a beneficial effect on AD. Infant formula comprising non-digestible oligosaccharides such as galacto-oligosaccharides and long chain fructo-oligosaccharides have been disclosed to reduce the incidence of atopic disease early in life (Moro et al, 2006, Arch Dis Child; 91:814-819.) The presence in the formulae of lactic acid producing bacteria, usually belonging to the genus Bifidobacterium or Lactobacillus, are disclosed to have beneficial effects in treating or preventing atopic dermatitis (Kalliomaki et al, 2001, Lancet 357:1076-1079; Chua et al, 2017, JPGN 65:102-106).

In order to determine whether an infant is at risk for developing atopic dermatitis currently the family history is taken into account, as mentioned above. But this method is subjective and not very precise; not all infants that are at risk are included; for example because the parental history on allergic disease is not known, not recalled or not realized. This may result in a considerable amount of infants developing atopic disease, in particular atopic dermatitis, that were initially not considered at risk and therefore did not get one of the above nutritional compositions that help in reducing the risk of developing atopic disease.

There is therefore a need to have a more precise and objective method to determine whether infants are at risk for developing atopic diseases, in particular atopic dermatitis which is the first step in the atopic march. Studies on the skin to determine biomarkers indicative for enhanced risk of atopic eczema are more objective but involve use of epidermal biopsies. While this is possible in adolescents or adults, it is not desired to obtain skin biopsies from infants and children, who have yet to develop AD. Therefore this method is not suitable. Additionally some reports mention analysis of umbilical cord blood to determine biomarkers such as IgE levels as risk factors for developing atopic disease. However, the value of the use of cord blood IgE as a predictive marker has been questioned by many and remains controversial with the lack of association with AD and allergy, poor sensitivity and low predictive values, as well as conflicting results amongst similar studies. Data from cord blood are heavily influenced by the status of the mother, for example by the nutritional Vitamin D status. Bergmann et al (1997, Clin Exp Allergy 27(7):752-760) concluded that the predictive capacity of parental history and cord blood IgE was not high enough to recommend them as screening instruments for primary prevention and that the majority of atopic manifestations and of sensitization occurred in infants without these risk factors of parental history and cord blood IgE levels. Furthermore, the practicality of analysing cord blood for biomarkers that predict AD is limited, as cord blood is now difficult to obtain for such purposes as privatised cord blood banking increases. Parents would prefer to bank their child's cord blood for use for future emergencies rather than analysing cord blood for predictors of AD which is a non-fatal disease.

### SUMMARY OF THE INVENTION

The inventors have found that the umbilical cord epithelium can be used as an easily accessible, non-invasive epidermal substitute for a predictive biomarker discovery. The umbilical cord is anatomically contiguous with the epidermis of the infant before birth, and is unwanted and discarded as medical waste. It was determined that the epidermis along the entire length of the cord, is representative for the immature skin. The presence and levels of five skin proteins was determined and correlated with the occurrence of atopic dermatitis later in life when the infant had reached an age of 3 months. It turned out that the level of three of the five biomarkers were significantly correlated with the occurrence of atopic dermatitis later in life, and the sensitivity improved when using a composite set of 3 biomarkers, and further improved when all 5 biomarkers were combined. All infants that developed atopic dermatitis later in life were detected with this method, resulting in an improved higher sensitivity compared with conventional risk assessment.

The presence of certain biomarkers in the umbilical cord epithelium thus enables to capture a higher proportion of the infants that are at risk for atopic dermatitis and the subsequent atopic diseases following atopic dermatitis such as allergy, rhinitis, and at an earlier stage and this enables an improved early nutritional intervention by administering adapted infant formula comprising ingredients known to prevent or reduce the risk for atopic dermatitis and the subsequent atopic disease, such as probiotics, prebiotics and/or hydrolysed proteins. Another advantage of the present invention is that an improvement in conducting clinical trials can be achieved, in particular a gain in efficiency can be achieved, as it allows correct identification and thus enrollment of a proper study population of infants at risk of developing atopic disease in for example clinical trials, which allows for a more efficient development, in particular in terms of time and costs, of new solutions for prevention and/or treatment of atopic disease.

### DETAILED DESCRIPTION OF THE INVENTION

The invention concerns a method for determining the risk of an infant to develop an atopic disease, wherein the method comprises:
a) determining in vitro the level of at least one biomarker protein in a sample comprising umbilical cord epithelial cells from the infant, and
b) comparing the level of the at least one biomarker protein to a reference value, and wherein a deviation in the level of the at least one biomarker protein in the sample compared to the reference value indicates an increased likelihood to develop the atopic disease, wherein the reference value is based on an average level of the same at least one biomarker protein in a control group that did not develop an atopic disease at the age of three months.

In a preferred embodiment, the method for determining the risk of an infant to develop an atopic disease further comprising providing an atopic disease customized diet for the infant in case of a deviation in the level of the at least one biomarker protein.

The invention concerns a method for determining the risk of an infant to develop an atopic disease, wherein the method comprises:
a) determining in vitro the level of at least one biomarker protein in a sample comprising umbilical cord epithelial cells from the infant, and
b) comparing the level of the at least one biomarker protein to a reference value, and wherein an increase in the level of the at least one biomarker protein in the sample compared to the reference value indicates an increased likelihood to develop the atopic disease, wherein the reference value is based on an average level of the same at least one biomarker protein in a control group that did not develop an atopic disease at the age of three months.

In a preferred embodiment, the method for determining the risk of an infant to develop an atopic disease further comprising providing an atopic disease customized diet for the infant in case of an increase in the level of the at least one biomarker protein.

Also the present invention concerns a method for customizing a diet for an infant at risk of developing an atopic disease, comprising
a) determining in vitro the level of at least one biomarker protein in a sample comprising umbilical cord epithelial cells from the infant, and
b) comparing the level of the at least one biomarker protein to a reference value,

and in case of a deviation in the level of the at least one biomarker protein in the sample compared to the reference value providing an atopic disease customized diet for the infant, wherein the reference value is based on an average level of the same at least one biomarker protein in a control group that did not develop an atopic disease at the age of three months.

Also the present invention concerns a method for customizing a diet for an infant at risk of developing an atopic disease, comprising
a) determining in vitro the level of at least one biomarker protein in a sample comprising umbilical cord epithelial cells from the infant, and
b) comparing the level of the at least one biomarker protein to a reference value,

and in case of an increase in the level of the at least one biomarker protein in the sample compared to the reference value providing an atopic disease customized diet for the infant, wherein the reference value is based on an average level of the same at least one biomarker protein in a control group that did not develop an atopic disease at the age of three months.

The invention also concerns a method of treatment of atopic disease in an infant by measuring for the presence of a deviation in the level of at least one biomarker protein in a sample comprising umbilical cord epithelial cells from the infant and treating the atopic disease by administering an atopic disease customized diet if an increase level of the at least one biomarker protein is found.

The invention also concerns a method for reducing the risk of developing of atopic disease in an infant by measuring for the presence of a deviation in the level of at least one biomarker protein in a sample comprising umbilical cord epithelial cells from the infant and if an increase in the level of the at least one biomarker protein is found administering an atopic disease customized diet thereby reducing the risk the infant develops atopic disease.

The invention also concerns a method of treatment of atopic disease in an infant by measuring for the presence of an increased level of at least one biomarker protein in a sample comprising umbilical cord epithelial cells from the infant and treating the atopic disease by administering an atopic disease customized diet if an increase level of the at least one biomarker protein is found.

The invention also concerns a method for reducing the risk of developing of atopic disease in an infant by measuring for the presence of an increased level of at least one biomarker protein in a sample comprising umbilical cord epithelial cells from the infant and if an increase in the level of the at least one biomarker protein is found administering an atopic disease customized diet thereby reducing the risk the infant develops atopic disease.

In a preferred embodiment, in the methods according to the present invention, the at least one biomarker protein is selected from the group consisting of loricrin, GATA-3, and kallikrein-7. More preferably in the methods according to the invention, the level of loricrin, the level of GATA-3, and the level of kallikrein-7 is determined and wherein an increase in the level of each of loricrin, GATA-3, and kallikrein-7 in the sample compared to the reference value of the same protein indicates an increased risk to develop the atopic disease.

In a further preferred embodiment, in the methods according to the present invention in addition to determining in vitro the level of at least one biomarker protein in a sample comprising umbilical cord epithelial cells from the infant wherein the at least one biomarker protein is selected from the group consisting of loricrin, GATA-3, and kallikrein-7, further the level of a biomarker protein selected from fillagrin and involcrin is determined, preferably the level of fillagrin and involcrin is determined, in vitro in a sample comprising umbilical cord epithelial cells from the infant and wherein an increase in the level of fillagrin and/or involcrin in the sample compared to the reference value of the same protein indicates an increased likelihood to develop the atopic disease, wherein the reference value is based on an average level of the same biomarker protein in a control group that did not develop an atopic disease at the age of three months.

Biomarkers/Assays

In its broadest sense, the invention concerns the use of a protein or a combination of proteins from umbilical cord epithelial cells from an infant as a marker for a predisposition to develop atopic disease in the infant. In one embodiment the protein from umbilical cord epithelial cells from an infant is GATA 3. In one embodiment the protein from umbilical cord epithelial cells from an infant is kallikrein-7 (KLK7). In one embodiment the protein from umbilical cord epithelial cells from an infant is loricrin. In one embodiment the protein from umbilical cord epithelial cells from an infant is fillagrin. In one embodiment the protein from umbilical cord epithelial cells from an infant is involcrin. In one embodiment the combination of proteins from umbilical cord epithelial cells GATA 3, kallikrein-7 and loricrin. In one embodiment the combination of proteins from umbilical cord epithelial cells GATA 3, kallikrein-7, loricrin and fillagrin. In one embodiment the combination of proteins from umbilical cord epithelial cells GATA 3, kallikrein-7, loricrin, and involcrin. In one embodiment the combination of proteins from umbilical cord epithelial cells GATA 3, kallikrein-7, loricrin, fillagrin and involcrin.

GATA-3 is a transcription factor with two conserved zinc finger motifs that bind to DNA consensus sequence (A/T)GATA(A/G). It is expressed in the developing nervous system, the embryonic kidney, inner ear, eye, skin and thymus but is found mainly in the hematopoietic system. In hematopoietic cells, GATA-3 is expressed by cells of T, natural killer (NK) and NKT lineages and is significantly up-regulated in hematopoietic cells that differentiate along the Th2 lineage. In skin, GATA-3 is expressed in the epidermis and the inner root sheath of the hair follicle where it regulates the hair follicle's inner root cell lineage and maintains the growth of postnatal hair. GATA-3 is well-known for its roles in the immune system where it plays a key role in T cell commitment and the development of Th2 immunity. It is the master regulator of Th2 cell differentiation, and the predominant regulator of Th2 cytokine expression. Expression of Th2 cytokines IL-4, IL-5 and IL-13 which are mediators of allergic inflammation, are regulated via chromatin remodeling when GATA-3 binds to multiple promoter sites of the Th2 cytokine locus. Corresponding with its role in promoting a Th2 skewed immune response, GATA-3 is found to be up-regulated in various allergies such as asthma and allergic rhinitis with an increased number of GATA-3 positive cells detected in patients with these conditions. Apart for its main role as a major regulator of the immune system, GATA-3 has also been shown to play important roles in epidermal barrier acquisition, with particular importance in the terminal stages of epidermal differentiation and desquamation via kallikrein 1 activation. GATA-3 was also found to regulate the biosynthesis of lipids essential for the maintenance of epidermal barrier integrity. Taken together, deficiency in GATA-3 contributes to various defects in proper epidermal terminal differentiation and lipid synthesis as described earlier, leading to a dysfunctional epidermal barrier, possibly contributing to AD pathogenesis.

Kallikrein related peptidase 7 (KLK7) is a chymotrypsin-like serine protease found in the epidermis which functions to cleave corneodesmosomal proteins as part of normal epidermal desquamation, contributing to maintenance of proper epidermal homeostasis and function. In transgenic mice, overexpression of KLK7 has been found to result in chronic itchy dermatitis, which is similar to chronic AD in humans. Stimulation of various inflammatory cytokines, such as Th2 cytokines IL-4 and IL-13 overexpressed in AD, significantly induced KLK7 expression in normal human epidermal keratinocytes compared to stimulation by Th1 and Th17 cytokines KLK7 has also been reported to degrade enzymes involved in lipid processing required in the maintenance of a proper epidermal barrier, leading to a dysfunctional epidermal barrier which contributes to AD pathogenesis.

Loricirn (LOR) is a glycine, serine and cysteine rich protein expressed in the granular layer of the epidermis. It is one of the main components of the cornified envelope, accounting for 70-85% of its total protein mass. In the epidermis, LOR gets crosslinked with other LOR molecules and cornified envelope proteins such as small proline rich proteins, keratins and FLG by transglutaminases. Also, LOR-deficient mice experience epidermal barrier dysfunction with compensatory upregulation of involucrin (IVL) and other small proline rich proteins which gets incorporated into the cornified envelope, highlighting the importance of LOR as an essential component of the cornified envelope and for the maintenance of a functional epidermal barrier.

Fillagrin (FLG) is expressed initially as profilaggrin contained in keratohyalin granules by differentiating keratinocytes in the granular layer. During terminal differentiation, profilaggrin gets dephosphorylated and cleaved to form FLG which aggregate keratin filaments in the granular and lower layers of the stratum corneum, promoting the collapse of cells, forming flattened corneocytes. At the surface of the stratum corneum, FLG gets degraded into free amino acids and are subsequently metabolised to form natural moisturizing factors (NMFs) essential for epidermal hydration. Small quantities of FLG, however, do not undergo degradation, but instead get integrated into the cornified envelope.

Involucrin (IVL) is a lysine, glysine and glutamine rich protein expressed early on during the formation of the cornified envelope. It forms the initial scaffold, allowing binding of other cornified envelope proteins via disulfide and Nε-(γ-glutamyl)lysine isopeptide bonds; and lipids via covalent bonds during the process of cornified envelope formation. Keratins are the main structural proteins in keratinocytes. In the proliferative basal layer, K5 and K14 expression dominates, with K1 and K10 being expressed later on during cornification, as keratinocytes undergo terminal differentiation moving upwards towards the stratum corneum, replacing previously established K5/K14 intermediate filament network. Together with FLG which aggregates the keratin filaments, keratin-FLG complexes which make up 80-90% of protein mass of the epidermisl, serve as a scaffold upon which other cornified envelope proteins gets crosslinked to during cornified envelope formation.

In a preferred embodiment, the level of biomarker protein refers to the level of the biomarker protein normalized to the level of glyceraldehyde 3-phosphate dehydrogenase (GAPDH-EC 1.2.1.12), which preferably simultaneously is determined and set at 1.

The reference value is the level of biomarker protein in the healthy reference group, which is the group of infants that have not developed atopic disease at the age of 3 months.

In a preferred embodiment, in the methods according to the present invention, the level of a biomarker protein is increased if the level of the biomarker protein normalized with regard to the level of glyceraldehyde 3-phosphate dehydrogenase (GAPDH) for loricrin ≥6.040, for GATA-3≥0.220, for kallikrein-7≥0.350, for fillagrin≥0.098 and/or for involcrin≥6.040. Preferably the level of the biomarker protein normalized with regard to the level of glyceraldehyde 3-phosphate dehydrogenase (GAPDH) for loricrin≥6.040 and for GATA-3≥0.220 and for kallikrein-7≥0.350. More preferably the level of the biomarker protein normalized with regard to the level of glyceraldehyde 3-phosphate dehydrogenase (GAPDH) for loricrin≥6.040 and for GATA-3≥0.220 and for kallikrein-7≥0.350 and for fillagrin≥0.098. More preferably the level of the biomarker protein normalized with regard to the level of glyceraldehyde 3-phosphate dehydrogenase (GAPDH) for loricrin≥6.040 and for GATA-3≥0.220 and for kallikrein-7≥0.350 and for involcrin≥6.040. More preferably the level of the biomarker protein normalized with regard to the level of glyceraldehyde 3-phosphate dehydrogenase (GAPDH) for loricrin≥6.040 and for GATA-3≥0.220 and for kallikrein-7≥0.350 and for fillagrin≥0.098 and for for involcrin≥6.040.

Procedures for determining the most optimal cut off value of a biomarker in order to achieve the most optimal sensitivity, specificity, and reduced outcomes of false positive and false positive values are known in the art. Typically these involve a ROC curve (receiver operating characteristic curve), which is a graphical plot that illustrates the diagnostic ability of a binary classifier system as its discrimination threshold is varied. The ROC curve is created by plotting the true positive rate (TPR) against the false positive rate (FPR) at various threshold settings. The true-positive rate is also known as sensitivity. The false-positive rate is also known as the fall-out or probability of false alarm and can be calculated as (1−specificity). The ROC curve is thus the sensitivity as a function of fall-out. Reference is further made to the experimental example.

Procedures for determining protein levels in cells are known. Preferably determining protein level is carried out involving a detection method, preferably a detection spectrometry based detection method, such as for example HPLC or LC/MS or a chromogenic assay. Alternatively or additionally determining protein level can involve or an antibody based detection method, such as ELISA, protein immunoprecipitation, immuno-electrophoresis, Western blot, protein immunostaining, RIA. A highly suitable method for determining protein levels involves Western blot analysis.

The umbilical cord epithelium is delicate and fragile and should be handled with care.

Nutritional Composition

The present invention also concerns a nutritional composition comprising ingredients that prevent or help to reduce the risk of developing atopic disease, preferably comprising at least one selected from the group consisting of hydrolysed protein, lactic acid producing bacteria and non-digestible oligosaccharides for use in preventing atopic disease in an infant, comprising
a) determining in vitro the level of at least one biomarker protein in a sample comprising umbilical cord epithelial cells from the infant, and
b) comparing the level of the at least one biomarker protein to a reference value

and in case of a deviation in the level of the at least one biomarker protein in the sample compared to the reference value administering the nutritional composition to the infant, wherein the reference value is based on an average level of the same at least one biomarker protein in a control group that did not develop an atopic disease at the age of three months.

In the context of this embodiment, it is noted that a deviation in the level of the at least one biomarker protein in the sample compared to the reference value indicates an increased likelihood to develop atopic disease.

The present invention also concerns a nutritional composition comprising at least one selected from the group consisting of hydrolysed protein, lactic acid producing bacteria and non-digestible oligosaccharides for use in preventing atopic disease in an infant, comprising
a) determining in vitro the level of at least one biomarker protein selected from the group consisting of loricrin, GATA-3, and kallikrein-7, in a sample comprising umbilical cord epithelial cells from the infant, and
b) comparing the level of the at least one biomarker protein to a reference value

and in case of an increase in the level of the at least one biomarker protein in the sample compared to the reference value administering the nutritional composition to the infant, wherein the reference value is based on an average level of the same at least one biomarker protein in a control group that did not develop an atopic disease at the age of three months.

In the context of this embodiment, it is noted that an increase in the level of the at least one biomarker protein in the sample compared to the reference value indicates an increased likelihood to develop atopic disease.

In a preferred embodiment of the use of nutritional composition according to the invention, the level of loricrin, GATA-3, and kallikrein-7 is increased.

In a further preferred embodiment of the use of nutritional composition according to the invention, in addition to determining in vitro the level of at least one biomarker protein in a sample comprising umbilical cord epithelial cells from the infant wherein the at least one biomarker protein is selected from the group consisting of loricrin, GATA-3, and kallikrein-7, further the level of a biomarker protein selected from fillagrin and involcrin is determined, preferably the level of fillagrin and involcrin is determined, in vitro in a sample comprising umbilical cord epithelial cells from the infant and wherein the level of fillagrin and/or involcrin, preferably the level of both, is increased in the sample compared to the reference value of the same biomarker protein, wherein the reference value is based on an average level of the same biomarker protein in a control group that did not develop an atopic disease at the age of three months.

In a preferred embodiment of the use of nutritional composition according to the invention, the level of a biomarker protein is increased if the level of the biomarker protein normalized with regard to the level of glyceraldehyde 3-phosphate dehydrogenase (GAPDH) for loricrin≥6.040, for GATA-3≥0.220, for kallikrein-7≥0.350, for fillagrin≥0.098 and/or for involcrin≥6.040. Preferably the level of the biomarker protein normalized with regard to the level of glyceraldehyde 3-phosphate dehydrogenase (GAPDH) for loricrin≥6.040 and for GATA-3≥0.220 and for kallikrein-7≥0.350. More preferably the level of the biomarker protein normalized with regard to the level of glyceraldehyde 3-phosphate dehydrogenase (GAPDH) for loricrin≥6.040 and for GATA-3≥0.220 and for kallikrein-7≥0.350 and for fillagrin≥0.098. More preferably the level of the biomarker protein normalized with regard to the level of glyceraldehyde 3-phosphate dehydrogenase (GAPDH) for loricrin≥6.040 and for GATA-3≥0.220 and for kallikrein-7≥0.350 and for involcrin≥6.040. More preferably the level of the biomarker protein normalized with regard to the level of glyceraldehyde 3-phosphate dehydrogenase (GAPDH) for loricrin≥6.040 and for GATA-3≥0.220 and for kallikrein-7≥0.350 and for fillagrin≥0.098 and for for involcrin≥6.040.

In a preferred embodiment, in the methods according to the present invention or of the use of the nutritional composition according to the invention the atopic disease customized diet comprises at least one of the group consisting of hydrolysed protein, lactic acid producing bacteria and non-digestible oligosaccharides.

The nutritional composition for use according to the invention (hereafter also referred to as the present composition or the composition) can be used as a nutritional composition, nutritional therapy, nutritional support, as a medical food, as a food for special medical purposes or as a nutritional supplement. The present composition is preferably an enteral (oral) composition. The composition is administered orally to, or intended to be administered orally to, a subject in need thereof, in particular to children and infants, including toddlers, preferably infants or young children typically with an age of 0-36 months, more preferably infants 0-12 months of age, most preferably 0-6 months of age. Thus, in some embodiments, the present composition is an infant formula, follow-on formula or young child formula (also referred to as growing-up milk), preferably it is an infant formula or follow-on formula, most preferably an infant formula. The term ‘infant formula’ is well-defined and controlled internationally and consistently by regulatory bodies. In particular, CODEX STAN 73-1981 “Standard For Infant Formula and Formulas For Special Medical Purposes Intended for Infants” is widely accepted. It recommends for nutritional value and formula composition, which require the prepared milk to contain per 100 ml not less than 60 kcal (250 kJ) and no more than 70 kcal (295 kJ) of energy. FDA and other regulatory bodies have set nutrient requirements in accordance therewith.

Preferably, the present enteral, preferably nutritional composition is for providing the daily nutritional requirements to a human, in particular for administration to, in particular for feeding, humans, in particular infants. The nutritional composition is not human milk.

In order to meet the caloric requirements of the infant, the present enteral composition preferably comprises 50 to 200 kcal/100 ml liquid, more preferably 60 to 90 kcal/100 ml liquid, even more preferably 60 to 75 kcal/100 ml liquid. This caloric density ensures an optimal ratio between water and calorie consumption. The osmolarity of the present composition is preferably between 150 and 420 mOsmol/l, more preferably 260 to 320 mOsmol/l. The low osmolarity aims to reduce the gastrointestinal stress.

Preferably, the present enteral composition is in a liquid form, preferably with a viscosity below 35 mPa·s, more preferably below 6 mPa·s as measured in a Brookfield viscometer at 20° C. at a shear rate of 100 s-1. Suitably, the present enteral composition is in a powdered from, which preferably can be reconstituted with water to form a liquid, or in a liquid concentrate form, which should be diluted with water. When the present enteral composition is in a liquid form, the preferred volume administered on a daily basis is in the range of about 80 to 2500 ml, more preferably about 450 to 1000 ml per day.

The composition according to the invention preferably comprises a lipid component, preferably a lipid component suitable for infant nutrition as known in the art. The lipid component of the present composition preferably provides 2.9 to 6.0 g, more preferably 4 to 6 g per 100 kcal of the composition. When in liquid form, the composition preferably comprises 2.1 to 6.5 g lipid per 100 ml, more preferably 3.0 to 4.0 g per 100 ml. Based on dry weight the present infant or follow on formula preferably comprises 12.5 to 40 wt % lipid, more preferably 19 to 30 wt %.

The composition according to the invention may comprise further proteinaceous material. In the context of the present invention the additional “protein” or “proteinaceous material” or “protein equivalents” encompasses proteins, peptides, free amino acids and partially or extensively hydrolysed proteins. The composition according to the present invention preferably contains less than 1 wt % intact mammalian (cow)'s milk protein. The composition may comprise an additional protein component selected from the group consisting of free amino acids, hydrolysed whey protein and proteins from other sources such as soy, pea, rice, collagen or the like, in intact form, in partially hydrolysed form, and/or in extensively hydrolysed form.

The present composition preferably contains at least 50 wt % protein component derived from non-human milk, more preferably at least 90 wt %, based on dry weight of total protein.

The present composition preferably contains 4 to 25%, more preferably 5 to 20%, more preferably 7 to 16%, most preferably 7 to 12% protein, based on total calories. The present composition, when in liquid form, preferably contains 0.5 to 6.0 g, more preferably 0.8 to 3.0 g, even more preferably 1.0 to 2.5 g of protein per 100 ml. The present composition preferably comprises at least 7.0 wt %, more preferably at least 8.0 wt %, most preferably at least 9 or at least 10 wt % protein based on dry weight of the total composition. Preferably, the present composition comprises at most 40 wt %, more preferably at most 15 wt %, preferably at most 20 wt % of protein based on dry weight of the total composition.

The composition may comprise digestible carbohydrate(s). Typically, digestible carbohydrates that are known in the art to be suitable for use in infant nutritional compositions are used, for example selected from digestible polysaccharides (e.g. starch, maltodextrin), digestible monosaccharides (e.g. glucose, fructose), and digestible disaccharides (e.g. lactose, sucrose). Particularly suitable is lactose and/or maltodextrin. In one embodiment, the composition does not comprise lactose.

The digestible carbohydrate component preferably comprises at least 60 wt % lactose based on total digestible carbohydrate, more preferably at least 75 wt %, even more preferably at least 90 wt % lactose based on total digestible carbohydrate.

Hydrolysed Protein

In one embodiment, the nutritional composition for use according to the present invention comprises hydrolysed protein. Preferably, the hydrolysed protein or proteinaceous material does not evoke an allergic reaction or is hypoallergenic, such as free amino acids and hydrolysed protein. The composition preferably comprises hydrolysed whey protein, preferably partially hydrolysed whey proteins. Such a protein component helps is reducing the risk for developing an atopic disease, in particular atopic dermatitis.

The composition according to the present invention preferably contains less than 1 wt % intact mammalian (cow)'s milk protein. The composition may comprise an additional protein component selected from the group consisting of free amino acids, hydrolysed whey protein and proteins from other sources such as soy, pea, rice, collagen or the like, in intact form, in partially hydrolysed form, and/or in extensively hydrolysed form.

The present composition preferably contains at least 50 wt % protein component derived from non-human milk, more preferably at least 90 wt %, based on dry weight of total protein. The present composition preferably contains at least 50 wt % hydrolysed protein component derived from non-human milk, more preferably at least 90 wt %, based on dry weight of total protein. Preferably the composition comprises at least 90 wt. % hydrolysed milk protein, preferably partially hydrolysed milk protein, based on total protein.

The protein hydrolysate (i.e. hydrolyzed proteins) dare preferably derived from mammalian milk, preferably milk from a species of the genus Bos, Bison, Bubalus or Capra, more preferably from genus Bos, most preferably from cow's milk (Bos taurus). In a preferred embodiment, the peptides are derived from whey protein. The nutritional composition preferably comprises at least 50 wt %, more preferably at least 70 wt %, even more preferably at least 95 wt % of hydrolysed whey protein based on total protein. A suitable source is a mixture of acid whey protein and demineralised sweet whey protein. Acid whey and sweet whey are commercially available. Sweet whey is the by-product of rennet-coagulated cheese and comprises caseinoglycomacropeptide (CGMP), and acid whey (also called sour whey) is the by-product of acid-coagulated cheese, and does not contain CGMP. Suitable sources for the whey protein are demineralised whey (Deminal, Friesland Campina, the Netherlands) and/or whey protein concentrate (WPC80, Friesland Campina, the Netherlands). The whey protein preferably comprises acid whey, more preferably at least 50 wt %, more preferably at least 70 wt % acid whey, based on total whey protein. Acid whey has an improved amino acid profile compared to sweet whey protein.

Hydrolysis may be achieved using a mixture of microbial endopeptidases and exopeptidases Preferably a mixture of an endoprotease and exoprotease is employed. The composition preferably comprises less than 10 wt %, preferably less than 6 wt % of peptides or proteins with a size of 5 kDa or above, based on total protein. It is preferred that more than 1 wt % of peptides or proteins present in the composition has a size of 1 kDa or above, based on total protein, more preferably at least 5 wt %, more preferably at least 10 wt %, based on total protein. The size distribution of the peptides in the protein hydrolysate can be determined by means of size exclusion high pressure liquid chromatography as known in the art. Saint-Sauveur er al. “Immunomodulating properties of a whey protein isolate, its enzymatic digest and peptide fractions” Int. Dairy Journal (2008) vol. 18(3) pages 260-270 describes an example thereof. In short, the total surface area of the chromatograms is integrated and separated into mass ranges expressed as percentage of the total surface area. The mass ranges are calibrated using peptides/proteins with a known molecular mass.

Lactic Acid Producing Bacteria

In one embodiment, nutritional composition for use according to the invention comprises lactic acid producing bacteria. The composition preferably comprises a strain of lactic acid producing bacterium species, which helps in preventing or treating atopic diseases, preferably atopic dermatitis. The bacterium strain is preferably a probiotic. Suitable lactic acid producing bacteria include strains of the genus Bifidobacteria (e.g. B. breve, B. longum, B. infantis, B. bifidum), Lactobacillus (e.g. L. acidophilus, L. paracasei, L. johnsonii, L. plantarum, L. reuteri, L. rhamnosus, L. casei, L. lactis), and Streptococcus (e.g. S. thermophilus). Bifidobacterium breve and Bifidobacterium longum are especially suitable lactic acid producing bacteria.

The nutritional composition for use according to the invention preferably comprises Bifidobacterium, preferably Bifidobacterium breve. The composition preferably comprises a strain of lactic acid-producing bacterium belonging to the genus Bifidobacterium, preferably to the species Bifidobacterium breve. The B. breve preferably has at least 95% identity of the 16 S rRNA sequence when compared to the type strain of B. breve ATCC 15700, more preferably at least 97% identity (Stackebrandt & Goebel, 1994, Int. J. Syst. Bacteriol. 44:846-849). Suitable B. breve strains may be isolated from the faeces of healthy human milk-fed infants. Typically, these are commercially available from producers of lactic acid bacteria, but they can also be directly isolated from faeces, identified, characterised and produced. According to one embodiment, the present composition contains a B. breve selected from the group consisting of B. breve Bb-03 (Rhodia/Danisco), B. breve M-16V (Morinaga), B. breve R0070 (Institute Rosell, Lallemand), B. breve BR03 (Probiotical), B. breve BR92) (Cell Biotech), DSM 20091, LMG 11613, YIT4065, FERM BP-6223 and CNCM I-2219. B. breve can be B. breve M-16V and B. breve CNCM 1-2219, most preferably B. breve M-16V. B. breve 1-2219 was published in WO 2004/093899 and was deposited at the Collection Nationale de Cultures de Microorganisms, Institute Pasteur, Paris, France on 31 May 1999 by Compagnie Gervais Danone. B. breve M-16V was deposited as BCCM/LMG23729 and is commercially available from Morinaga Milk Industry Co., Ltd.

The lactic acid producing bacterium may be present in the composition at any suitable concentration, preferably in a therapeutically effective amount or “amount effective for treating” in the context of the invention. Preferably, the lactic acid producing bacterium strain is included in the present composition in an amount of 10⁴-10¹³ cfu per g dry weight of the composition, preferably 10⁵-10¹¹ cfu/g, most preferably 10⁶-10¹⁰ cfu/g.

Non-Digestible Oligosaccharides

In a preferred embodiment, the present composition comprises one or more non-digestible oligosaccharides [NDO]. The presence of NDO help in preventing or treating atopic diseases, preferably atopic dermatitis.

Advantageously and most preferred, the non-digestible oligosaccharide is water-soluble (according to the method disclosed in L. Prosky et al, J. Assoc. Anal. Chem 71: 1017-1023, 1988) and is preferably an oligosaccharide with a degree of polymerisation (DP) of 2 to 200. The average DP of the non-digestible oligosaccharide is preferably below 200, more preferably below 100, even more preferably below 60, most preferably below 40.

The non-digestible oligosaccharide is preferably a prebiotic. It is not digested in the intestine by the action of digestive enzymes present in the human upper digestive tract (small intestine and stomach). The non-digestible oligosaccharide is fermented by the human intestinal microbiota. For example, glucose, fructose, galactose, sucrose, lactose, maltose and the maltodextrins are considered digestible. The non-digestible oligosaccharide raw materials may comprise monosaccharides such as glucose, fructose, fucose, galactose, rhamnose, xylose, glucuronic acid, GalNac etc., but these are not part of the non-digestible oligosaccharides. The non-digestible oligosaccharide is preferably selected from the group consisting of fructooligosaccharide, non-digestible dextrin, galactooligosaccharide, xylooligosaccharide, arabino-oligosaccharide, arabinogalacto-oligosaccharide, glucooligosaccharide, glucomanno-oligosaccharide, galactomanno-oligosaccharide, mannan-oligosaccharide, chito-oligosaccharide, uronic acid oligosaccharide, sialyloligosaccharide, and fucooligosaccharide, and mixtures thereof, preferably fructo-oligosaccharides. Examples of sialyloligosaccharide are 3-sialyllactose, 6″ sialyllactose, sialyllacto-N-tetraoses, disialyllactoNtertraoses. Examples of fucooligosaccharides are (un)sulphated fucoidan oligosaccharides, 2′fucosyllactose, 3′ fucosyllactose, lacto-N-fucopentaose I, II, III, LNDH, lactodifucotetraose, lacto-N difucohexaose I, II.

One suitable type of oligosaccharide is a short-chain oligosaccharide which has an average degree of polymerisation of less than 10, preferably at most 8, preferably in the range of 2-7. The short-chain oligosaccharide preferably comprises galacto-oligosaccharides and/or fructo-oligosaccharides (i.e. scGOS and/or scFOS). In one embodiment, the composition comprises galacto-oligosaccharides, preferablybeta-galacto-oligosaccharides, preferably trans-galacto-oligosaccharides. The galacto-oligosaccharides preferably have an average degree of polymerisation in the range of 2-8, preferably 3-7, i.e. are short-chain oligosaccharides in the context of the invention. (Trans)galactooligosaccharides are for example available under the trade name Vivinal® GOS (Friesland Campina Domo Ingredients, Netherlands), Bimuno (Clasado), Cup-oligo (Nissin Sugar) and Oligomate55 (Yakult). The composition preferably comprises short-chain fructo-oligosaccharides and/or short-chain galacto-oligosaccharides, preferably at least short-chain fructo-oligosaccharides. Fructooligosaccharides may be inulin hydrolysate products having an average DP within the aforementioned (sub-) ranges; such FOS products are for instance commercially available as Raftilose P95 (Orafti) or with Cosucra.

Another suitable type of oligosaccharide is long-chain fructo-oligosaccharides (IcFOS) which has an average degree of polymerisation above 10, typically in the range of 10-100, preferably 15-50, most preferably above 20. A particular type of long-chain fructo-oligosaccharides is inulin, such as Raftilin HP.

The present composition may contain a mixture of two or more types of non-digestible oligosaccharides, most preferably a mixture of two non-digestible oligosaccharides. In case the NDO comprises or consists of a mixture of two distinct oligosaccharides, one oligosaccharide may be short-chain as defined above and one oligosaccharide may be long-chain as defined above. Most preferably, short-chain oligosaccharides and long-chain oligosaccharides are present in a weight ratio short-chain to long-chain in the range of 1:99-99:1, more preferably 1:1-99:1, more preferably 4:1-97:3, even more preferably 5:1-95:5, even more preferably 7:1-95:5, even more preferably 8:1-10:1, most preferably about 9:1.

In one embodiment, the composition comprises at least two of fructo-oligosaccharides and/or galacto-oligosaccharides. Suitable mixtures include mixtures of long-chain fructo-oligosaccharides with short-chain fructo-oligosaccharides or with short-chain galacto-oligosaccharides, most preferably long-chain fructo-oligosaccharides with short-chain fructo-oligosaccharides.

The present composition preferably comprises 0.05 to 20 wt % of said non-digestible oligosaccharides, more preferably 0.5 to 15 wt %, even more preferably 1 to 10 wt %, most preferably 2 to 10 wt %, based on dry weight of the present composition. When in liquid form, the present composition preferably comprises 0.01 to 2.5 wt % non-digestible oligosaccharide, more preferably 0.05 to 1.5 wt %, even more preferably 0.25 to 1.5 wt %, most preferably 0.5-1.25 wt %, based on 100 ml.

When the non-digestible oligosaccharide is a mixture, the averages of the respective parameters are used for defining the present invention.

The combination of a NDO and a lactic acid producing bacterium as defined here above is also referred to as a “synbiotic”. The presence of therapeutically effective amounts of the NDO together with the lactic acid-producing bacterium are believed to further improve the effect in preventing or treating atopic diseases, preferably atopic dermatitis. Preferred combination is a strain of Bifidobacterium, preferably B. breve, together with galacto-oligosaccharides and/or fructo-oligosaccharides.

Other Components

The composition may further comprise long chain polyunsaturated fatty acids (LC-PUFA). LC-PUFA are fatty acids wherein the acyl chain has a length of 20 to 24 carbon atoms (preferably 20 or 22 carbon atoms) and wherein the acyl chain comprises at least two unsaturated bonds between said carbon atoms in the acyl chain. More preferably the present composition comprises at least one LC-PUFA selected from the group consisting of eicosapentaenoic acid (EPA, 20:5 n3), docosahexaenoic acid (DHA, 22:6 n3), arachidonic acid (ARA, 20:4 n6) and docosapentaenoic acid (DPA, 22:5 n3), preferably DHA, EPA and/or ARA. Such LC-PUFAs have a further beneficial effect on reducing the risk for atopic diseases, including atopic dermatitis.

The preferred content of LC-PUFA in the present composition does not exceed 15 wt. % of total fatty acids, preferably does not exceed 10 wt. %, even more preferably does not exceed 5 wt. %. Preferably the present composition comprises at least 0.2 wt. %, preferably at least 0.25 wt. %, more preferably at least 0.35 wt. %, even more preferably at least 0.5 wt. % LC-PUFA of total fatty acids, more preferably DHA. The present composition preferably comprises ARA and DHA, wherein the weight ratio ARA/DHA preferably is above 0.25, preferably above 0.5, more preferably 0.75-2, even more preferably 0.75-1.25. The weight ratio is preferably below 20, more preferably between 0.5 and 5. The amount of DHA is preferably above 0.2 wt %, more preferably above 0.3 wt %, more preferably at least 0.35 wt %, even more preferably 0.35-0.6 wt % on total fatty acids.

Human Subjects

The human subjects or population targeted are preferably humans subjects, preferably infants, at risk of developing atopic diseases, such as atopic dermatitis, allergy, preferably milk protein allergy, allergic rhinitis and asthma. The nutritional composition for use according to the present invention may be used in human subjects of 0-3 years of age. In a preferred embodiment, the nutritional composition is for use in infants from 0-12 months. In a preferred embodiment, the nutritional composition is for use in infants from 0-6 months, more preferably in infants from 0-3 months.

In yet a further preferred embodiment, the nutrition composition is for use in infants directly after determining an increase following comparing the level of the biomarker proteins to a reference value as explained herein, in particular under step b), or as a first nutrition next to or after human milk consumption or as an alternative to human milk consumption.

Atopic Disease

The present invention concerns determining the risk of an infant to develop an atopic disease and also the present invention concerns preventing atopic disease in an infant or reducing the risk that an infant develops atopic disease. In a preferred embodiment according to the present invention, the atopic disease is atopic dermatitis.

Atopic dermatitis (AD), also referred to as atopic eczema or allergic eczema, is a chronic inflammatory skin disease commonly affecting infants and young children. It is a relapsing-remitting disorder characterized by intense pruritus and recurrent eczematous lesions which appear during the flares. This disease usually presents itself during early infancy within the first few months of life and in childhood, with some exceptions that begin only during adolescence or in adulthood. Improvements are usually seen in 70% of cases over time and most cases will usually resolve in late childhood. Severe cases, however, may persist into or relapse in adolescence and adulthood. AD is believed by many to be the first step in the atopic march, resulting in asthma and allergic rhinitis in most of the afflicted individuals later in life. Clinically, AD is the first indicator of allergy. The earliest signs of AD are the dryness and roughness of skin as AD lesions do not typically appear during the first month of life. Beyond the first month, eczematous lesions appear primarily on the face, on the cheeks and chin, with sparing of the nose and paranasal area; the scalp; trunk; and extensor surfaces of limbs in infants. In children, adolescents and adults, lesions appear mainly on the neck and flexural areas such as inside of the elbows and behind the knees. In addition to the flexures, AD lesions can also typically present itself at the wrists, ankles, eyelids, hands and feet in adolescents and adults. Regardless of age, intense pruritus is often associated with AD. AD can present itself at any age and can be categorized into three groups based on the age of onset: infantile AD, childhood AD and adolescent or adulthood AD. Of those afflicted with AD, 45% developed the disease within the first six months of life; 60% within the first year of life and 95% before the age of five. Generally, an earlier age of onset was found to be associated to a more severe and persistent AD phenotype. Methods to determine AD are known in the art. AD can be determined by a physician. One method to assess the severity of AD is the SCORAD (severity scoring of atopic dermatitis; Consensus Report of the European Task Force on Atopic Dermatitis. Dermatology 1993; 186:23-31).

### EXAMPLES

### Example 1: Characterisation of the Umbilical Cord Epithelium

Materials and Methods

Umbilical cords (n=15) were collected in total from normal healthy infants at birth with informed consent from mothers prior delivery. Three cords were collected with placenta attached and cut into three sections: nearer fetus, middle and nearer placenta. Representative pieces from each of the three sections were collected, with a piece frozen and another fixed in formalin and paraffin embedded for subsequent histological analyses. For the rest of the umbilical cords collected (n=12), only a single piece of the umbilical cord from an unknown, random location along the cord was obtained. These umbilical cord samples from unknown locations along the cord were fixed in formalin and paraffin embedded for subsequent analyses.

The samples should be handled carefully. After delivery the entire cord was cut from placenta end to umbilical cord clamp. The pieces of umbilical core (2.5 cm each) were placed into a 50 mL Falcon tube filled with 10% Neutral Buffered Formalin (at least 20× volume of tissue) and tubes were sealed with parafilm to ensure no leakages.

An intact mouse umbilical cord sample (n=1) from a healthy pup with epidermis and placenta attached at opposite ends was collected for histology. The entire piece was fixed in formalin, paraffin-embedded and sectioned longitudinally for subsequent histological analyses.

For frozen samples, the umbilical cord was embedded in optimal cutting temperature (OCT) compound consisting of polyethylene glycol and polyvinyl alcohol at room temperature and frozen in liquid nitrogen before being cryo-sectioned for histological analysis. For formalin fixed paraffin embedded samples (FFPE), the umbilical cord was fixed in formalin then paraffin embedded and sectioned for histological analysis. Immunofluorescence (IF) and immunohistochemistry (IHC) staining to visualize the proteins of interest were carried out on frozen and paraffin-embedded sections respectively.

Hematoxylin and eosin (H&E) staining was carried out to determine morphology and structure of frozen and FFPE sections. For FFPE sections, sections were dewaxed by incubation in xylene and rehydrated by bringing the sections through decreasing percentages of ethanol (100%, 90%, 80% and 70%) prior to H&E staining proper. For H&E staining, nuclei were stained with hematoxylin for 5 minutes and rinsed with running tap water. Sections are then differentiated with 1% acid alcohol for 30 seconds and blued with Scott's tap water (blueing solution) for 2 minutes, with rinsing under running tap water between steps. Next, cytoplasm was stained with Eosin Y dye (Sigma), and the tissue dehydrated through increasing percentages of alcohol and lastly incubated in xylene prior mounting. Dried slides of H&E stained tissues were examined under Zeiss microscopic imager to determine the morphology and structure of the tissues.

Frozen umbilical cord samples embedded in OCT were cyro-sectioned and mounted on Superfrost plus slides (Leica). The sections were then incubated with primary antibodies overnight at 4° C. and detected with Alexa Fluor 488 fluorophore-conjugated secondary goat anti-mouse (GAM) or goat anti-rabbit (GAR) antibodies (Invitrogen). The stained slides were counter-stained with 4, 6-diamidino-2-phenylindole (DAPI) for visualization of nuclei. Expression patterns of proteins of interest were examined under using Zeiss microscopic imager (Zeiss) and qualitative scoring of the expression levels and patterns was performed.

FFPE umbilical cord samples were sectioned and mounted on Superfrost plus slides (Leica). The slides were placed in slide holders and heated at 50° C. in a dry oven overnight to facilitate attachment of tissue. Prior to IHC staining, the sections were dewaxed in xylene and rehydrated. Next, antigens of tissue sections were retrieved by heat exposure using 1× antigen exposing citrate buffer pH6 solution (Dako) overnight, endogenous peroxidases in the tissue sections were quenched by incubation with 1% hydrogen peroxide for 30 minutes, and non-specific sites in the tissue section were then blocked with 10% goat's serum for 20 minutes. These sections were then incubated with primary antibodies overnight at 4° C. and antigens visualized by probing with secondary antibodies conjugated with HRP polymer using the DAKO EnVision™+System (Dako). Nuclei were counterstained with hematoxylin and the sections dehydrated and mounted for microscopic visualization using Zeiss microscopic imager (Zeiss).

Using histological methods it was found that the umbilical cord (UC) epithelium exhibits variable phenotypes (thin monolayer, thicker monolayer, bi-multilayer regions, transition zones between monolayer and bi-multilayer and invaginations with thicker bi multilayer regions. The UC epithelium is delicate and fragile and should be handled with care. The heterogeneity of the UC phenotypes extends through the whole umbilical cord. There was no gradual change from simple to stratified between epidermis and umbilical cord (data not shown).

It was determined whether epidermal associated protein expression profiles on skin and the umbilical cord (epidermis) were comparable. Indeed this is the case, as is shown in Table 1. The UC epithelium of all acquired samples were stained with antibodies targeting various epithelial biomarkers (keratins) and the expression profile of these biomarkers were compared to the epidermis as reference to determine the nature of the UC epithelium. Besides keratins, the profile of several biomarkers of various parts of the skin (e.g. epidermis: FLG, LOR, IVL; basement membrane: collagen VII; dermis: vimentin) were investigated to determine the degree of similarity between the UC epithelium and its underlying Wharton's jelly connective tissue to the epidermis and dermis of the skin. Epidermal related proteins such as KLK7, CLDN1 and ECAD were tested to determine likeness of UC epithelium with the epidermis, since these proteins are also expressed in the epidermis. Table 1 below summarizes the expression profile of the various biomarkers in the UC epithelium in relation to the epidermis, a classical example of stratified epithelia.

TABLE 1
Comparison of epidermal associated protein
expression profiles of UC and skin

| | | Present in | |
|---|---|---|---|
| Protein | Description | UC epithelium | Epidermis |
| Keratin 7/8 | Simple epithelial marker | + | − |
| Keratin 18 | Simple epithelial marker | + | − |
| Keratin 19 | Simple epithelial marker | + | − |
| Keratin 14 | Stratified epithelial marker | + | + |
| Keratin 10 | Stratified epithelial marker | + | + |
| Keratin 6 | Hyperproliferation marker | + | + |
| Keratin 16 | Hyperproliferation marker | + | + |
| Filaggrin | Cornified envelope protein | −/+ | + |
| Loricrin | Cornified envelope protein | + | + |
| Involucrin | Cornified envelope protein | + | + |
| Collagen VII | Basement membrane marker | + | + |
| Vimentin | Mesenchymal cell marker | − | − |
| | | (+ in | (+ in |
| | | Wharton's | dermis) |
| | | jelly) | |
| Kallikrien-7 | Epidermal serine protease | + | + |
| Claudin-1 | Tight junction protein | + | + |
| E-cadherin | Adherens junction protein | + | + |

To characterize the UC epithelium, several staining procedures on frozen and FFPE collected UCs were carried out. IF staining of selected pieces of frozen umbilical cords sections from unknown location along the length of the UC, systematic IHC staining of representative FFPE sections from the three UC sub-sections: fetal end, middle and placental end to determine the distribution of keratins and epidermal proteins along the length of the UC, and IHC staining of a representative piece of UC from different UC samples, were carried out.

The UC epithelium was found to express: stratified epithelia associated keratins K10 and K14; simple epithelia associated keratins K7/8, K18 and K19; and hyper-proliferation associated keratins K6 and K16. The distribution patterns of stratified epithelial keratins K10 and K14 were found to be similar in both the UC epithelium and epidermis. K10, a suprabasal layer biomarker, expressed only in cell layers above the basal layer in the epidermis, was similarly expressed only in the upper cell layers of the multilayer regions of the UC epithelium. K14, a marker of the basal layer of the epidermis, was found to be expressed throughout the whole UC epithelium, in both the monolayer and multilayer regions of the UC epithelium. However, unlike the epidermis which does not express any simple epithelial keratins, the UC epithelium was found to express simple epithelial biomarkers K7, K8, K18 and K19 throughout the UC epithelium. The expression of K7, K8 and K18 in UC epithelium was much less than K19. K6 and K16 hyper-proliferation-associated proteins were also found to be expressed throughout the UC epithelium. Amongst the three epidermal cornified envelope barrier proteins investigated, IVL and LOR were both expressed throughout the UC epithelium, by all epithelial cells in both the monolayer and multilayer regions of the UC. Unlike IVL, which was expressed uniformly strongly throughout the UC epithelium, LOR was expressed to a greater extent in the superficial layers of the multilayer regions than in the lower layers.

Like the epidermis, the UC epithelium also expresses basement membrane biomarker collagen VII. The Wharton's jelly of the UC, like the dermis of skin, also expresses vimentin, a marker of mesenchymal cells. Similarly, epidermal protease KLK7, tight junction protein CLDN1 and adherens junction protein ECAD which are found in the epidermis were also found to be expressed in the UC epithelium. Expression of KLK7 was found to be greater in the superficial layer of the UC epithelium while CLDN1 and ECAD on the other hand, were found to be expressed throughout the whole UC epithelium.

In conclusion, the UC epithelium of both the cross-section and representative longitudinal sections along the length of the whole UC was characterized. The UC epithelium represents a unique transitional type epithelium which is neither simple nor stratified per se as it concurrently expresses both simple (K7, K8, K18, K19) and stratified epithelial (K10, K14, LOR, IVL) markers along the whole length of the UC. The UC epithelium, especially the stratified multilayer regions can be considered to be representative of early skin that is not completely matured. The large similarities in epidermal differentiation marker expression between the two tissues are an indication that the UC epithelium, which is ethically and easily obtainable by non-invasive means, can be used as a surrogate of the epidermis to replace epidermal biopsies from infants or young children for predictive biomarker research and in monitoring the events of early onset AD.

Furthermore, it would not matter which part of the UC the samples were obtained from. Instead, we feel that it would be more important to utilize histology as an initial quality check step to ensure the integrity of the tissue collected prior to using these samples for downstream analyses.

### Example 2: Identification of Potential Early Predictive Biomarkers of Atopic Dermatitis in the Umbilical Cord

A total of 1247 subjects were recruited into the GUSTO birth cohort. A subset of Chinese GUSTO subjects (n=42) were selected in this umbilical cord protein biomarkers pilot study. Cases (n=20) were subjects that had AD at three months. Controls (n=22) were subjects that had no AD at three months and no family AD history. Table 2 below summarizes the demographics and clinical characteristics of the umbilical cord protein biomarkers study cohort. Demographic information and clinical follow-up data were obtained through questionnaires completed at three months, six months, 12 months, 15 months, 18 months and 36 months. Skin prick tests were conducted at 18 months and 36 months. SCORAD was recorded when AD was present during the 18th and 36th month clinical visits.

TABLE 21
Demographic and clinical characteristics of umbilical
cord protein biomarkers study cohort (n = 42)

| Characteristic | AD (n = 20) | | Non-AD (n = 22) | |
|---|---|---|---|---|
| Gender (n, %) | | | | |
| Male | 11 | (45.8) | 13 | (54.2) |
| Female | 9 | (50.0) | 9 | (50.0) |
| Gestational age | 39.01 | (1.144) | 38.38 | (0.860) |
| (weeks) (Mean, sd) | | | | |
| Mode of delivery | | | | |
| Vaginal | 12 | (40.0) | 18 | (60.0) |
| Cesarean | 8 | (66.7) | 4 | (33.3) |
| Family AD history (n, %) | | | | |
| Yes | 10 | (100.0) | 0 | (0.0) |
| No | 10 | (31.2) | 22 | (68.8) |
| Family allergy history (n, %) | | | | |
| Yes | 13 | (65.0) | 7 | (35.0) |
| No | 7 | (31.8) | 15 | (68.2) |
| Skin prick test at | | | | |
| 18^{th} month (n, %) | | | | |
| Positive (Food or HDM) | 7 | (77.8) | 2 | (22.2) |
| Negative (Food or HDM) | 10 | (34.5) | 19 | (65.5) |
| SCORAD (Mean, sd) (n = 4) | 19.62 | (6.815) | | NA |

Allergy refers to asthma, AD, allergic rhinitis. Family refers to parents and siblings. SCORAD, SCORing Atopic Dermatitis. SCORAD mean and standard deviation readings were based on four subjects as only four subjects had active AD during 18th month clinical visit.

Whole cord protein lysates made from frozen umbilical cords (n=42) collected at birth and corresponding clinical data of healthy infants and infants with early AD.

Proteins from the whole umbilical cord were extracted by homogenizing crushed umbilical cord samples in RIPA buffer and protease inhibitor mix (Roche) using a homogenizer. Once the tissue is completely disrupted, debris was spun down at 3000 rpm at 4° C. for 2 minutes. Supernatant from the mixture was then spun down again at 13200 rpm at 4° C. for 10 minutes to remove remaining insoluble materials. The final resulting supernatant was then quantified and stored at −80° C. for downstream Western blot analyses.

Protein concentration of the samples was determined using the bicinchoninic acid (BCA) protein assay kit. (Pierce). 25 μl of diluted bovine serum albumin standards were prepared from serial dilutions. The BCA working reagent containing 50 parts of BCA solution and 1 part of 4% of cupric sulphate solution were added to the standards and protein samples and mixed briefly. This mixture was then incubated at 37° C. for 30 minutes and later, absorbance was read at 540 nm. A standard curve was plotted using absorbance values of diluted bovine serum albumin standards and used to determine the protein concentration of each protein sample.

20 μg of protein samples were loaded into pre-cast Any kD™ Mini-PROTEAN® TGX™ gels (Bio-rad), separated by sodium dodecyl sulphate-polyacrylamide gel electrophoresis and transferred to polyvinyl difluoride (PVDF) membranes. After transfer, the PVDF membranes was blocked with 5% non-fat milk prior to incubation with primary antibody overnight at 4° C. and then probed with horse radish peroxidase (HRP)-conjugated secondary antibody in the dilutions listed in Table 8 below. Bound secondary antibodies were detected via enhanced chemiluminescence using Immun-Star HRP chemiluminescent substrate kit (Bio-rad). Protein bands were visualized using the LICOR Odyssey imager (LI-COR) with its intensities were evaluated by densitometry measured using the LICOR Image Studio Software Version 2.1 (LI-COR), and later quantified after normalization against GAPDH.

Western blot experimental data were analysed and visualized using GraphPad Prism 6 for Windows (GraphPad Software). Normality was assumed based on visual analysis of histograms and conducting the Shapiro-Wilks test. Quantification of Western blot protein bands was determined with densitometry. All protein densities from test groups were normalized against the respective GAPDH band intensity. Data was expressed as mean±standard error of mean. Mann Whitney U test, *P<0.05, **P<0.01 (AD: n=20, non-AD: n=22).

Receiver-operating characteristic (ROC) analysis using SPSS 16.0 for Windows (SPSS Inc.) was performed to determine the cut-off threshold for each individual biomarker. Biomarker cut-off thresholds were determined by calculating Youden's index (J) with the formula: sensitivity+specificity−1. The point corresponding to the maximum value of J indicates the optimal cut-off threshold of a biomarker when equal importance is given to both sensitivity and specificity 358. This maximum value of J corresponding to top left most corner of the ROC curve which indicates a cut-off value that gives the highest true positive rate and lowest false positive rate.

Experimental values greater than the calculated cut-off threshold represent a positive test result corresponding to a positive predicted outcome.

Composite biomarker indexes or panels of combinations of identified potential biomarkers were obtained via two methods: black box modelling and risk-score modelling. Multiple binary logistic regression analysis using SPSS 16.0 for Windows (SPSS Inc.) was employed for both modeling methods.

In black box modeling, the relationships between the biomarker variables are unknown and not accounted for. In this modeling method, raw biomarker levels derived from Western blot densitometric analysis for each biomarker were combined by multiple binary logistic regression to give a combined composite marker score ranging from 0 to 1 (predicted probabilities). A combined composite marker cut-off threshold was calculated based on these combined predicted probabilities as described above. Experimental values greater than the combined composite marker cut-off threshold represent a positive test result corresponding to a positive predicted AD outcome.

In risk-score modeling, risk-scores in predicting AD derived from odds ratio calculated via multiple binary logistic regression analysis were assigned to each individual biomarker. In this modeling method, cut-off thresholds of each individual biomarker were first determined as described in above. Experimental values greater than the combined composite marker cut-off threshold represent a positive test result corresponding to a positive predicted outcome. Results of predicted outcomes for each individual biomarker were then combined to form two separate risk-score models via multiple binary logistic regression. The first model combines three (significantly different) biomarkers based on Mann Whitney U test and the second model combines all five tested biomarkers. A combined risk-score cut-off threshold was calculated based on the sum of risk-scores using the ROC analysis. For each positive test result of each individual biomarker which exceeds the biomarker cut-off threshold, the corresponding risk-score was given. Negative test result of the individual biomarker will be given a risk-score of zero. Risk-scores of all individual biomarkers given to each subject were then totaled and compared to a combined risk-score cut-off threshold. Calculated overall risk-score of each subject greater than the calculated combined risk-score cut-off threshold represent a positive test result corresponding to a positive prediction of AD.

Five factors: i) sensitivity, ii) specificity, iii) positive predictive value (PPV) iv) negative predictive value (NPV) and v) discriminatory power were compared to evaluate individual and composite biomarkers. Experimental values (derived from Western blot densitometry analyses) exceeding the cut-off threshold derived from ROC analyses indicated a positive prediction of AD. Predicted outcomes were then cross-tabulated against actual observed clinical outcomes obtained from clinical data. Both predicted and observed outcome numbers were used to calculate the sensitivity, the true positive rate, or the proportion of actual positives which are correctly identified; specificity, the true negative rate or the proportion of negatives which are correctly identified; positive predictive value, the proportion of positive results that are true positives; negative predictive value, the proportion of negative results that are true negatives.

ROC analysis using SPSS 16.0 for Windows (SPSS Inc.) was used to determine the discriminatory power of individual biomarkers in distinguishing AD and non-AD by computing the area under ROC (AUROC) curve. AUROC values between 0.50 and 0.60 regarded as a useless test; values between 0.60 and 0.70 regarded as poor test; values between 0.70 and 0.80 regarded as a fair test; values between 0.80 and 0.90 regarded as a good test; and values between 0.90 and 1 as an excellent test. P-values and 95% confidence intervals were calculated. P-values of less than 0.05 were regarded as statistically significant.

Western blot experimental data were analysed and visualized using GraphPad Prism 6 for Windows (GraphPad Software). Normality was assumed based on visual analysis of histograms and conducting the Shapiro-Wilks test. Test of significance between AD cases and non-AD control groups were performed using Mann Whitney U test to identify the potential biomarkers which discriminate the AD cases and non-AD controls. P-values of less than 0.05 were regarded as statistically significant. *, P<0.05, **, P<0.01 and ***, P<0.001 indicate a statistically significant difference between AD and non-AD groups.

Results Western blot was carried out to determine FLG, IVL, LOR, GATA3 and KLK7 expression in the umbilical cord. FLG (˜26 kD), IVL (˜120 kD), LOR (˜52 kD), GATA3 (˜48 kD) and (pro)-KLK7 (˜38 kD) were detected in all umbilical cord samples. Densitometry analysis of the Western blot revealed LOR (p=0.010), GATA-3 (p=0.008) and KLK7 (p=0.015) levels were significantly differentially regulated in infants that developed AD at three months compared to infants that did not, the values in AD subjects being higher. FLG and IVL expression were also higher in infants that developed AD at three months compared to infants that did not, but these correlations showed a trend (p<0.10)

TABLE 2
Median values of biomarker protein density levels
in umbilical cords of infants with and without AD

| | AD (n = 20) | Non-AD (n = 22) | |
|---|---|---|---|
| Biomarker | (Median) | (Median) | P-value |
| FLG | 0.123 | 0.08809 | 0.060 |
| IVL | 0.1556 | 0.1061 | 0.099 |
| LOR | 7.72 | 5.645 | 0.010 * |
| GATA-3 | 0.2651 | 0.1723 | 0.008 ** |
| KLK7 | 0.3681 | 0.3445 | 0.015 * |

P-values from Mann Whitney U test.
* p < 0.05,
** p < 0.01

Each protein biomarker was evaluated separately to determine its potential as a predictive biomarker of AD by performing the ROC analysis (ROC=Receiver operator characteristics, a graph wherein the sensitivity is plotted against 1−specificity and calculation of AUROC (the area under the ROC curve), sensitivity, specificity, positive predictive values (PPV) and negative predictive values (NPV) (See Table 4).

TABLE 4
FLG, IVL, LOR, GATA-3 and KLK7 as potential predictive
biomarkers of AD

| | | AUROC | Sensitivity | Specificity | PPV | NPV |
|---|---|---|---|---|---|---|
| Biomarker | Cut-off | (95% CI) | (%) | (%) | (%) | (%) |
| Filaggrin | ≥0.098 | 0.670 | 75.0 | 63.6 | 65.2 | 73.7 |
| | | (0.504-0.837) | | | | |
| Involucrin | ≥0.091 | 0.650 | 90.0 | 45.5 | 60.0 | 83.3 |
| | | (0.482-0.818) | | | | |
| Loricrin | ≥6.040 | 0.730* | 80.0 | 54.5 | 61.5 | 75.0 |
| | | (0.578-0.881) | | | | |
| GATA-3 | ≥0.220 | 0.736** | 70.0 | 68.2 | 66.7 | 71.4 |
| | | (0.582-0.891) | | | | |
| Kallikrien-7 | ≥0.350 | 0.718* | 75.0 | 63.6 | 65.2 | 73.7 |
| | | (0.561-0.876) | | | | |

PPV, Positive predictive value; NPV, Negative predictive value. *p < 0.05, **p < 0.01.

LOR, GATA-3 and KLK7 levels are fair biomarkers as they have AUROC values between 0.7 to 0.8.

TABLE 5
Risk-scores for each biomarker of both risk-score models
(5 biomarker risk-score model and 3 biomarker risk-score model)

| Biomarker | Cut-off | 5 Biomarkers | 3 Biomarkers |
|---|---|---|---|
| FLG | ≥0.098 | 2 | — |
| IVL | ≥0.091 | 11 | — |
| LOR | ≥6.040 | 10 | 6 |
| GATA-3 | ≥0.220 | 6 | 6 |
| KLK7 | ≥0.350 | 3 | 4 |

| | | |
|---|---|---|
| Total | 32 | 16 |

Composite biomarkers indexes or panels of combined individual markers created by black box and risk-score modeling were evaluated to determine its potential as a predictor of AD by performing the ROC analysis and calculation of AUROC, sensitivity, specificity, PPVs and NPVs (Table 6).

TABLE 6
Evaluation of composite markers derived from black box and
risk-score modeling

| | | AUROC | Sensitivity | Specificity | PPV | NPV |
|---|---|---|---|---|---|---|
| Biomarker | Cut-off | (95% CI) | (%) | (%) | (%) | (%) |
| Black box | ≥0.322 | 0.802** | 95.0 | 54.5 | 65.5 | 92.3 |
| (3 markers) | | (0.671-0.934) | | | | |
| Black box | ≥0.488 | 0.841*** | 75.0 | 86.4 | 83.3 | 79.2 |
| (5 markers) | | (0.720-0.962) | | | | |
| Risk-score | ≥7 | 0.816*** | 75.0 | 72.7 | 71.4 | 76.2 |
| (3 markers) | | (0.687-0.945) | | | | |
| Risk-score | ≥14 | 0.880*** | 100.0 | 54.5 | 66.7 | 100.0 |
| (5 markers) | | (0.777-0.982) | | | | |

PPV, Positive predictive value; NPV, Negative predictive value. **p < 0.01, ***p < 0.001.

The three-protein black box-derived composite biomarker could predict correctly those who developed AD at three months 95% of the time (5% false negative rate) and predict correctly those who will not 54.5% of the time (45.5% false positive rate). Among those with positive predictions, the probability of developing AD was 65.5% and among those with negative predictions, the probability of not developing AD was 92.3%.

The five-protein black box-derived composite biomarker could predict correctly those who developed AD at three months 75% of the time (25% false negative rate) and predict correctly those who will not 86.4% of the time (14.6% false positive rate). Among those with positive predictions, the probability of developing AD was 83.3% and among those with negative predictions, the probability of not developing AD was 79.2%.

The three-protein risk-score-derived composite biomarker could predict correctly those who developed AD at three months 75% of the time (25% false negative rate) and predict correctly those who will not 72.7% of the time (27.3% false positive rate). Among those with positive predictions, the probability of developing AD was 71.4% and among those with negative predictions, the probability of not developing AD was 76.2%.

The five-protein risk-score-derived composite could predict correctly those who developed AD at three months 100% of the time (no false negatives) and predict correctly those who will not 54.5% of the time (45.5% false positives). Among those with positive predictions, the probability of developing AD was 66.7% and among those with negative predictions, the probability of not developing AD was 100%. Combining the individual biomarkers to form composite biomarkers indices increased the discriminatory value of the biomarkers in predicting AD as shown by the significant increase of AUROC values from between 0.6 to 0.7 (FLG and IVL) and between 0.7 to 0.8 (LOR, GATA-3 and KLK7) (Table 6) to between 0.8 to 0.9. Based on AUROC values determined from the ROC curve plot, the composite markers determined by combining either the three significant biomarkers LOR, GATA-3 and KLK7 or all five tested biomarkers, were good predictive biomarkers of AD.

Combining individual biomarkers to form composite biomarkers is more realistic in mirroring the multifactorial nature of AD and also improved the predictive power of the biomarkers shown by increase in calculated AUROC values. The composite biomarker derived from the combination of all five biomarkers obtained via risk-score modeling gave the best performance as it has the best discrimination power between those who develop AD at three months and those who do not. This risk-score test consisting of the five biomarker composite however was a high sensitivity-low specificity test, with sensitivity value of 100%, specificity value at 54.5%, PPV of 66.7% and NPV of 100%. This composite biomarker could predict correctly those who would eventually develop AD all the time, missing out none of those who have the disease (no false negatives), predict correctly those who will not develop AD 54.5% of the time and falsely predicting that 45.5% would develop AD when they will not (false positives). The test had a false positive prediction rate of 45.5%. It is still clinically acceptable due to the fact that the proposed nutritional compositions of prevention or treatment have little to no side effects.

These biomarkers, as in the claims, in particular the composite biomarkers, more particular the composite biomarkers with the five biomarkers that resulted in a 100% sensitivity of the test is promising, allowing to identify all those infants with high risk of developing AD, so that this high risk group can be enlisted into early preventive treatment programs including the administration of specific nutritional compositions as in the present claims. Such programs may curtail the development of AD, potentially modifying the course of the atopic march and impeding further development of allergies later on in life.

1. A method for determining the risk of an infant to develop an atopic disease, wherein the method comprises:
a) determining in vitro the level of at least one biomarker protein from umbilical cord epithelial cells in a sample comprising umbilical cord epithelial cells from the infant, and
b) comparing the level of the at least one biomarker protein to a reference value, and wherein an increase in the level of the at least one biomarker protein in the sample compared to the reference value indicates an increased likelihood to develop the atopic disease, wherein the reference value is based on an average level of the same at least one biomarker protein in a healthy reference group of infants that did not develop an atopic disease at the age of three months.

2. The method according to claim 1, further comprising providing an atopic disease customized diet for the infant in case of an increase in the level of the at least one biomarker protein.

3. A method for customizing a diet for an infant at risk of developing an atopic disease, comprising
a) determining in vitro the level of at least one biomarker protein from umbilical cord epithelial cells in a sample comprising umbilical cord epithelial cells from the infant, and
b) comparing the level of the at least one biomarker protein to a reference value, and in case of an increase in the level of the at least one biomarker protein in the sample compared to the reference value providing an atopic disease customized diet for the infant, wherein the reference value is based on an average level of the same at least one biomarker protein in a control group that did not develop an atopic disease at the age of three months.

4. The method according to claim 2, wherein the atopic disease customized diet comprises at least one of the group consisting of hydrolysed protein, lactic acid producing bacteria and non-digestible oligosaccharides.

5. The method according to claim 1, wherein the at least one biomarker protein is selected from the group consisting of loricrin, GATA-3, and kallikrein-7.

6. The method according to claim 1, wherein the level of loricrin, GATA-3, and kallikrein-7 is determined and wherein an increase in the level of each of loricrin, GATA-3, and kallikrein-7 in the sample compared to the reference value of the same protein indicates an increased risk to develop the atopic disease, preferably wherein the level of a biomarker protein is increased if the level of the biomarker protein normalized with regard to the level of glyceraldehyde 3-phosphate dehydrogenase (GAPDH) for loricrin≥6.040, for GATA-3≥0.220, for kallikrein 7≥0.350, for fillagrin≥0.098 and/or for involcrin≥6.040.

7. The method according to claim 6 wherein further the level of a biomarker protein from umbilical cord epithelial cells selected from fillagrin and involcrin, preferably both, is determined in vitro in a sample comprising umbilical cord epithelial cells from the infant and wherein an increase in the level of fillagrin and/or involcrin, preferably of both, in the sample compared to the reference value of the same protein indicates an increased likelihood to develop the atopic disease, wherein the reference value is based on an average level of the same biomarker protein in a control group that did not develop an atopic disease at the age of three months.

8. (canceled)

9. The method according to claim 1, wherein the atopic disease is atopic dermatitis.

10. A method for preventing atopic disease in an infant, the method comprising:
a) determining in vitro the level of at least one biomarker protein from umbilical cord epithelial cells selected from the group consisting of loricrin, GATA-3, and kallikrein-7, in a sample comprising umbilical cord epithelial cells from the infant, and
b) comparing the level of the at least one biomarker protein to a reference value and in case of an increase in the level of the at least one biomarker protein in the sample compared to the reference value;
administering a nutritional composition comprising at least one selected from the group consisting of hydrolysed protein, lactic acid producing bacteria and non-digestible oligosaccharides to the infant,
wherein the reference value is based on an average level of the same at least one biomarker protein in a control group that did not develop an atopic disease at the age of three months.

11. The method according to claim 10, wherein the level of loricrin, GATA-3, and kallikrein-7 is increased.

12. The method according to claim 10, wherein further the level of a biomarker protein from umbilical cord epithelial cells selected from fillagrin and involcrin, preferably both, is determined in a sample comprising umbilical cord epithelial cells from the infant and wherein the level of fillagrin and/or involcrin, preferably both, is increased in the sample compared to the reference value of the same biomarker protein, wherein the reference value is based on an average level of the same biomarker protein in a control group that did not develop an atopic disease at the age of three months.

13. The method according to claim 10, wherein the level of a biomarker protein is increased if the level of the biomarker protein normalized with regard to the level of glyceraldehyde 3-phosphate dehydrogenase (GAPDH) for loricrin≥6.040, for GATA-3≥0.220, for kallikrein 7≥0.350, for fillagrin≥0.098 and for involcrin≥6.040.

14. The method according to claim 10, wherein the atopic disease is atopic dermatitis.

15. The method according to claim 10, wherein the nutritional composition is an infant formula or follow on formula.

16. The method according to claim 10, wherein the infant has an age from 0-6 months, more preferably from 0-3.

17. The method according to claim 10, wherein the nutritional composition is administered directly after determining an increase following comparing the level of the biomarkers under step b) or as a first nutrition next to or after human milk consumption.

18. The method according to claim 3, wherein the atopic disease customized diet comprises at least one of the group consisting of hydrolysed protein, lactic acid producing bacteria and non-digestible oligosaccharides.

19. The method according to claim 3, wherein the at least one biomarker protein is selected from the group consisting of loricrin, GATA-3, and kallikrein-7.

20. The method according to claim 3, wherein the level of loricrin, GATA-3, and kallikrein-7 is determined and wherein an increase in the level of each of loricrin, GATA-3, and kallikrein-7 in the sample compared to the reference value of the same protein indicates an increased risk to develop the atopic disease, preferably wherein the level of a biomarker protein is increased if the level of the biomarker protein normalized with regard to the level of glyceraldehyde 3-phosphate dehydrogenase (GAPDH) for loricrin≥6.040, for GATA-3≥0.220, for kallikrein 7≥0.350, for fillagrin≥0.098 and/or for involcrin≥6.040.

21. The method according to claim 20, wherein further the level of a biomarker protein from umbilical cord epithelial cells selected from fillagrin and involcrin, preferably both, is determined in vitro in a sample comprising umbilical cord epithelial cells from the infant and wherein an increase in the level of fillagrin and/or involcrin, preferably of both, in the sample compared to the reference value of the same protein indicates an increased likelihood to develop the atopic disease, wherein the reference value is based on an average level of the same biomarker protein in a control group that did not develop an atopic disease at the age of three months.

22. The method according to claim 3, wherein the atopic disease is atopic dermatitis.
